(19) United States
(12) Patent Application Publication
(10) Pub. No.: US 2024/0008800 A1
(43) Pub. Date: Jan. 11, 2024

(54) EARBUD APPARATUS WITH INTEGRATION OF REAL TIME IN-THE-EAR ELECTROENCEPHALOGRAPHY AND ELECTRODE PORT THAT CAN SIMULTANEOUSLY PLAY AUDIO VIA SPEAKER HOUSING

(71) Applicant: Ryan Ahmed, Kew Gardens, NY (US)

(72) Inventors: Ryan Farzam Ahmed, Kew Gardens, NY (US); Shariar Huda, Jamaica, NY (US); Authoy Das, Jamaica, NY (US); Steffani Rajapaksha, Bayonne, NJ (US); Melani Shrestha, Elmhurst, NY (US); Alisha Karim, Queens, NY (US); Chloe Kan, Oakland Gardens, NY (US); Arun Chhugani, Bethlehem, PA (US); Chenginling Zheng, Flushing, NY (US); Genevive Chang, New York City, NY (US); Dang Nguyen, Wellington, FL (US); Pari Patel, Hagerstown, MD (US); Jinnat Mahmood, Jamaica, NY (US); Katherine Xiao, Watkinsville, GA (US); Lesley Osasogielyamu, Mount Vernon, NY (US); Mohammad Rahim, Jamaica, NY (US); Nana Adom Opare-Addo, Bronx, NY (US); Dhriti Rathod, Chambersburg, PA (US); Sakef Uddin, Jamaica, NY (US); Samiha Nasrin, Bronx, NY (US); Saqib Khan, Elmont, NY (US); Saifa Sowa, Hollis, NY (US); Tanvi Jammula, Ashburn, VA (US); Veda Kolli, Ballwin, MO (US); Wajiha Moin, Brooklyn, NY (US); William Yu, Bayside, NY (US); Deven Rozario, Flushing, NY (US)

(73) Assignee: Ryan Ahmed, (omitted)

(21) Appl. No.: 18/452,526

(22) Filed: Aug. 19, 2023

(51) Int. Cl. A61B 5/00; A61B 5/374; A61B 5/31; A61B 5/384; H04R 1/10; G06F 3/16

(52) U.S. Cl. CPC A61B 5/4088; A61B 5/374; A61B 5/31; A61B 5/0006; A61B 5/6817; A61B 5/384; H04R 1/1016; G06F 3/165

(57) ABSTRACT

An earbud system integrates real-time EEG monitoring with audio playback. Using specialized silicone electrodes, it captures and interprets EEG data, serving as a passive and continuous tool for screening neurological conditions. This allows for on-the-spot brain monitoring while retaining conventional earbud functions. It can particularly be used in medical scenarios, like open brain surgery, where traditional EEGs cannot be integrated during the operation itself. The system aids emotional balance, stress relief, and neurostimulation, bolstering brain health, mood enhancement, and relaxation. Its applications span wellness for therapeutic interventions and entertainment for tailored content using biometric feedback. This invention broadens traditional earbud capabilities, merging biometrics, health tracking, and customized audio suggestions.

### FIELD

The present invention relates to bio-physiological monitoring devices, specifically those designed for in-ear placement, enabling simultaneous electroencephalography (EEG) data acquisition and audio playback. Uniquely, the ear-centric device employs silicone, embedded with conductive filaments, as the electrode itself, integrated within the earbud design for efficient EEG data collection. Associated software not only processes the collected metrics for personalized music recommendations but also facilitates potential medical evaluations, including the detection of neurological irregularities. As such, the invention bridges the disciplines of audio engineering, biometric analysis, and health screening.

### BACKGROUND

Description of the Problem: Each year, the Centers for Disease Control and Prevention reports over 795,000 stroke cases in the U.S. This number is in addition to cases of other neurological conditions such as aneurysms and epilepsy. A challenge in addressing these conditions is the lack of widespread early detection methods. Electroencephalography (EEG) is a clinical tool used to record the brain's electrical activity. Current EEG equipment is based on older technology, primarily focusing on diagnostics. The cost of standard EEG devices can be high, which may limit their accessibility to some populations. The design of traditional EEG equipment also hinders its incorporation into everyday products like earbuds. An EEG mechanism that is affordable and can be integrated into commonly used items might offer an approach to increase the frequency and reach of early neurological screenings.

Description of the Related Art: The domain of biometrics, while seasoned in certain applications like electrocardiograms and biometric authentication systems, has not seen equivalent advancements when it comes to EEG technology. Traditional EEG diagnostics involve medical-grade equipment where electrodes are strategically positioned on the scalp. These electrodes detect signals from various brain regions, resulting in charts that professionals analyze. Originating in the early 20th century, these traditional EEG setups, despite their longevity, present challenges: they are often uncomfortable, unwieldy, time-consuming, and expensive.

Several contemporary inventions have attempted to modernize this practice. They've sought to design EEG devices that are compact and portable, often employing fewer electrodes and focusing on specific brain regions for enhanced utility. However, these modern adaptations are not devoid of limitations. A predominant challenge is the scalp-based electrode placement, which can be obtrusive and potentially affect brain activity readings. In stark contrast, the technology under discussion advocates for an in-ear EEG approach, steering away from conventional scalp-based systems. This shift addresses not only the discomfort associated with traditional electrode placement but also augments discreteness and portability.

Historical EEG devices' bulkiness and heft curtailed their portability, confining their use predominantly to controlled settings. This inherent design aspect hindered the versatility of potential EEG applications. Moreover, conventional EEG systems are sensitive to external disturbances. Factors like perspiration or movement can interfere with readings, leading to potential inaccuracies. This vulnerability can be likened to the distortion experienced on TV screens during adverse conditions, highlighting the significance of signal noise in EEG data. Additionally, the interpretation of such data typically requires specialized skills, further narrowing its accessibility.

Within the sphere of ear-based biometric wearables, conventional designs externally attach metallic or conductive components directly to the earbud ear-tip. Notably, these traditional systems don't employ the silicone itself as the electrode, where in such embodiments distinct portions of the whole earbud ear-tip can be demarcated to form electrode arrays with great surface area to inside the ear. Some earlier designs position electrodes outside the ear, using an externally attached hook. This arrangement alters the aesthetic of conventional consumer earbuds, making the embedded technology readily apparent. Given these recognized challenges, the presented technology aims to provide alternative solutions to address the limitations of current EEG methodologies.

Objective of the Invention: The primary objective of this invention is to provide a novel system that utilizes EEG technology embedded in earbud devices for the screening of brain abnormalities and monitoring neuroelectrophysiology. The silicone electrodes made with conductive filaments will capture data indicative of brain activity, enabling the early detection of abnormalities such as epileptic seizures, brain-related disorders, and other neurological irregularities. This invention allows affordable and efficient real-time monitoring on a user interface. The invention, consisting of an EEG-integrated earbud apparatus and silicone electrodes, facilitates the monitoring of neural biometrics. Through the use of these silicone electrodes, EEG data can be captured efficiently, assisting in the screening and intervention of anomalies before they begin to progress.

Brief Summary of the Invention: The invention described herein pertains to an earbud apparatus that integrates real-time in-the-ear electroencephalography (EEG) capabilities. At its core, the apparatus employs silicone embedded with conductive filaments, acting as electrodes, to capture EEG data. This technical design differs markedly from standard EEG equipment, which often utilizes external electrodes placed on the scalp. Instead, the present invention employs an in-ear methodology, with the electrode system situated within the earbud's eartip. A pivotal feature of this apparatus is its capacity to detect a comprehensive range of brainwave frequencies, encompassing alpha, beta, theta, delta, and gamma waves. These frequencies, indicative of various cognitive and emotional states, are captured through the silicone-conductive filament mix, ensuring a robust signal quality with minimal interference. A complementary component of this invention is the associated software that processes the acquired EEG data. Beyond merely documenting the EEG readings, the software is engineered to analyze the data for potential music curation, thereby aligning audio output with the user's detected mental state. This simultaneous acquisition and processing ability ensures real-time responsiveness to the user's biometric feedback. From a structural standpoint, the earbud apparatus is designed to offer dual functionality. While its primary function facilitates audio playback, the integrated electrode port concurrently captures EEG data. Such integration obviates the need for external EEG equipment, offering users an unobtrusive means of monitoring brain activity while engaging with audio content. Furthermore, the design considers potential partitioning of the conductive eartip, allowing for a grid formation. Such a configuration is anticipated to augment the surface area contact within the ear, enhancing the granularity of EEG data capture. The possibility of extending the conductive elements to the general ear hook, serving as an additional positive or ground electrode, showcases the intricate technical considerations for optimal data capture. In essence, this invention represents a convergence of biometric analysis and audio technology. Through meticulous design considerations, it combines the utility of traditional earbuds with the advanced capabilities of EEG equipment, heralding a new direction in personal audio and biometric monitoring devices.

Technical Terminology and Concepts: Electroencephalogram (EEG): A screening and diagnostic method employed to measure and record electrical activity generated by the brain. This is done using electrodes placed on the scalp, which capture fluctuations in voltage resulting from ionic current flows within the neurons of the brain.

Electrodes: Conductive devices or mediums designed to detect electrical signals. In the context of the present invention, these are integrated into earbuds to facilitate non-invasive capture of EEG data.

EEG-Integrated Earbuds: A novel integration of EEG technology into earbuds. This involves the incorporation of electrodes within the earbuds' structure, enabling the continuous capture of EEG data while performing the primary function of audio delivery.

Alpha, Beta, Theta, Delta, and Gamma Waves: Different classifications of brainwaves based on their frequencies. Each type is associated with specific mental states or functions and can provide insights into brain health and activity.

EEG Amplifier: A component responsible for enhancing the electrical signals captured by EEG sensors. It ensures that the signals are of sufficient strength and clarity for subsequent processing and analysis.

Amplitude: In the context of EEG, amplitude refers to the magnitude of the electrical signal or brainwave at any given point in time.

Frequency: Represents the number of oscillations or cycles of a wave per unit of time. In EEG, it helps in categorizing brain waves into types like Alpha, Beta, etc.

Shape and Duration: These are graphical representations and lengths of time, respectively, for specific brain waves or patterns. Variations in these parameters can offer insights into potential neurological anomalies or states of consciousness.

### SUMMARY

The Centers for Disease Control and Prevention documents more than 795,000 stroke cases and upwards of 100,000 Alzheimer's-related fatalities in the United States, annually based on current available metrics. These figures are worsened by the absence of early detection techniques for neurological disorders. Traditional earbud products have yet to incorporate conventional electroencephalography (EEG), and expanding the utilization of EEG technology can increase screening rates for brain abnormalities. The presented apparatus combines conductive electrode technology using an earbud eartip, resulting in a screening tool that measures neural activity. To ensure accurate EEG data collection, silicone electrodes are built around a mixture that includes conductive filaments that are adept at detecting essential alpha, beta, theta, and gamma brainwaves. This technology is facilitated through an exclusive brain-activity interface. Moreover, the design places a strong emphasis on comfort of the wearer and user experience. By integrating portable EEG screening in a passive and continuous method, the technology not only promotes familiarity among its users but also fosters a sense of audio and musical connectivity through shared listening experiences.

The present disclosure involves the integration of electroencephalography systems into an earbud, creating a minimally invasive screening tool for neurological conditions. The proposed invention includes conductive elements such as silicone eartips and electrodes embedded in an earbud apparatus; these elements would document oscillation waves from various lobes of the brain and are amplified through specific software such as an EEG amplifier. In other embodiments, the conductive ear tip can be partitioned into a grid formation, allowing the electrode arrays to have greater surface area within the ear tip itself. Having greater electrode arrays allows for more accurate EEG signal readings. Moreover, the general ear hook may be made out of conductive silicone elements as well to serve as a positive or ground electrode. The increased electrode coverage stemming from this arrangement enhances the granularity and accuracy of EEG signal capture. Through amplification, recorded brain waves are presented using various outputs, including PC and mobile software. Furthermore, key advantages of the present disclosure involve instantaneous monitoring of brain health while performing other tasks of a traditional earbud. Users are able to experience a multitude of capabilities from personalized music recommendation to real-time health oversight. Moreover, another advantage includes non-invasive techniques that capture EEG data only through the ear. As opposed to the proposed invention, current clinical EEG applications involve multiple electrodes being attached to the scalp.

The presented earbud apparatus combines the dual functionality of in-the-ear electroencephalography (EEG) data acquisition and audio playback. Through integrated electrodes, it captures and interprets EEG patterns, subsequently curating music playlists corresponding to detected moods. Beyond personal audio use, the invention finds potential applications in medical fields for early screening of brain abnormalities. For instance, the proposed invention has the ability to capture biometrics during surgical operations such as open or closed brain surgery, providing critical information during procedures. Other applications include uses in wellness sectors for mood-based therapeutic interventions, and in the entertainment industry for personalized content delivery based on real-time biometric feedback. This fusion of biometrics and audio technology broadens the applications of traditional earbuds, offering both recreational and health-monitoring benefits.

### BRIEF DESCRIPTION OF THE DRAWINGS

In the following portion relating to the detailed description, the embodiments of the present disclosure will be explained more in detail with reference to the example figures of the proposed invention shown in the drawings, which:

FIG. 1 depicts a front-view perspective of the EEG earbud system in the present disclosure.

FIG. 2 illustrates a rear-view perspective of the EEG earbud system in the present disclosure.

FIG. 3 delineates a side-view perspective of an individual wearing the EEG earbud system in present disclosure.

FIG. 4 portrays an aerial view of the acquisition of data through alpha, beta, gamma, and theta brainwaves.

FIG. 5 is an aerial view of the EEG circuit embedded in the EEG earbud system.

FIG. 6 displays a schematic diagram of the EEG earbud system once the earbud apparatus is placed into the ear canal and has optimal function.

### DETAILED DESCRIPTION

The present invention discloses an advanced earbud system equipped with non-obstructing EEG technology, designed to monitor brainwave activity in real time while simultaneously delivering high-quality audio experiences.

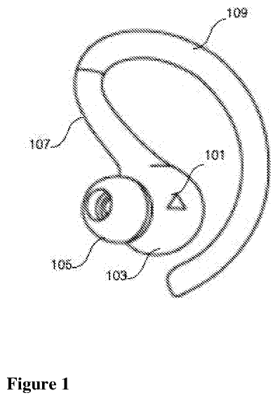

FIG. 1 provides a front-view representation of the EEG earbud system, highlighting its essential components. The 101—Ground Electrode is designed for direct contact with the user's ear. Its primary function is to detect EEG signals from various regions in the brain. As the reference point, this electrode plays a significant role in noise reduction, thereby enhancing the clarity of EEG signals.

Adjacent to the ground electrode is the 103—Body of the Earbud. This structure serves as the container for critical components of the EEG earbud system: including wiring, circuitry, and battery storage. It effectively links the components, ensuring integrated functionality of the earbud system. The 105—Silicone Ear Tip is designed to fit within the outer ear canal. Beyond its role in audio quality improvement by channeling sound waves, its conductive properties allow it to play an integral part in EEG data capture. Its conductivity, achieved through various methods such as the integration of conductive filaments, facilitates the transmission of EEG signals to the relevant circuitry. Some embodiments might incorporate a grid pattern on the eartip to increase the electrode arrays' surface area, aiming to enhance EEG reading accuracy. Further down in the same figure, the 107—Earbud Hook is highlighted. This component is designed to loop around the user's ear, providing stability. Added stability ensures proper positioning of 105 during EEG signaling. In some design variations, the hook incorporates conductive silicone elements, potentially enabling more extensive EEG data capture around the ear. Additionally, the 109—End of Earbud Hook contributes to the device's structural stability, especially during prolonged use.

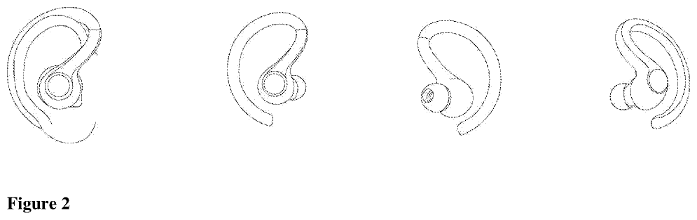

In FIG. 2 the rear view of the EEG earbud system is presented in FIG. 2. Central to this figure is the 103—Earbud Body, serving its role as the main structural component of the EEG earbud system. The 201—Speaker is showcased, indicating its function of transforming digital inputs into auditory outputs. This audio delivery occurs concurrently with the EEG data capture facilitated by the silicone eartip. Thus, users of the presented invention can perform tasks such as listening to music and conducting phone calls while the EEG detection technology occurs. Other components illustrated include the 203—Battery, a rechargeable unit responsible for powering the system. Additionally, the 205—Circuit provides a glimpse into the technical operations of the system. It illustrates the stages of EEG data acquisition, amplification, and subsequent digitization. With the support of efficient circuitry and noise-reduction techniques, this component emphasizes the system's dedication to maintaining data precision. Lastly, the 105—Eartip is revisited, reinforcing its dual function in refining audio delivery and capturing EEG data.

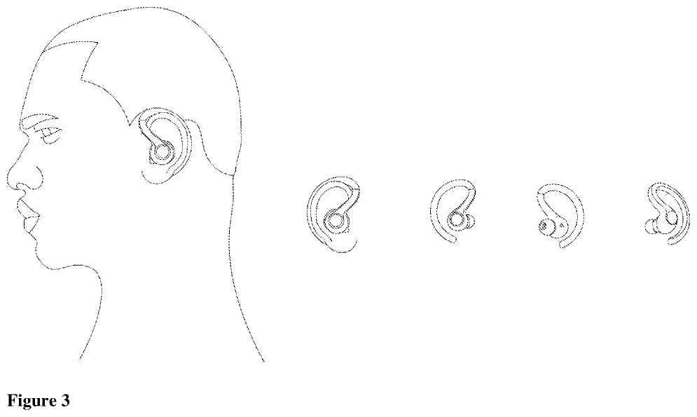

Earbud and Human Interaction in FIG. 3 illustrates a humanoid wearing the earbuds. The user, denoted by 301, benefits from audio outputs. The audio is generated by the earbud, as referred to by 303, equipped with audio drivers optimized for sound playback. The earbud also features a microphone for audio input purposes. While the user 301 performs the standard tasks of an earbud, EEG signals are detected and interpreted instantaneously. The design ensures that as the user engages with audio content, the device concurrently records EEG data, monitoring the user's brainwave activity.

Ground Electrode and Earbud Hook integrated within the earbud is the ground electrode, marked as 101, which contacts the user's ear. This interface is vital for data acquisition, focusing on EEG data from the brain's temporal lobe. An earbud hook, labeled 107, is incorporated to ensure the stability of the device and the consistent positioning of the ground electrode. Designed for endurance, this hook promotes continuous and stable usage. It's worth noting that certain models might employ conductive silicone in the earbud hook, allowing for additional electrode arrays not only within the ear canal but also around the ear's exterior. This approach broadens the EEG data capture domain, aiming for enhanced precision. The tail of this earbud hook, 109, is designed for a snug fit around the lower part of the ear, further stabilizing the device.

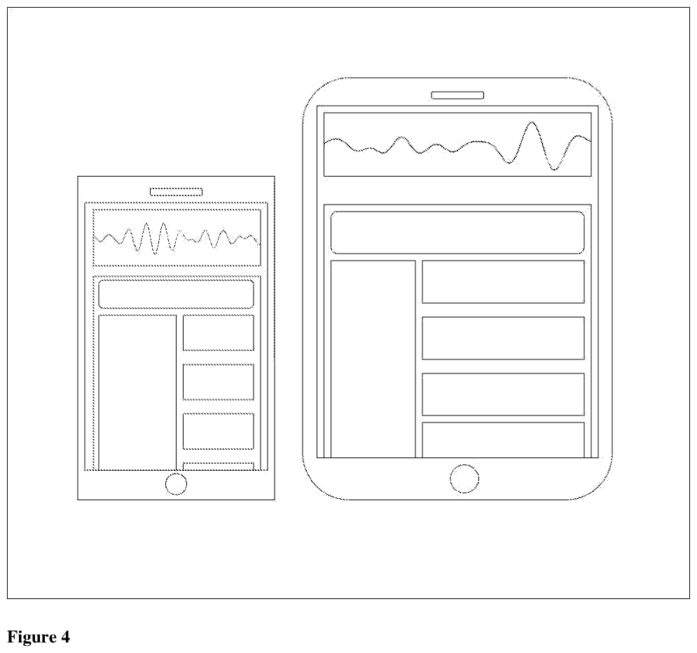

An aerial perspective in FIG. 4 visualizes the data acquisition process, capturing a range of brainwaves, notably alpha, beta, gamma, and theta frequencies. This visualization underscores the earbud's capability to monitor a comprehensive spectrum of brainwave activities. Software Integration for Devices in FIG. 4 delves into the software interaction potential of the EEG earbud system. Capable of wireless data transmission, the system can interface with Human-Machine Interface (HMI) software on various devices. Indications 401 and 403 point towards compatibility with mobile and tablet software, respectively. Once the data is relayed, both the mobile and tablet software can graphically represent EEG waves, as denoted by 405 and 407. This real-time visualization offers users insights into their brain activity. Additionally, 409 and 411 outline potential and alternative interface displays that incorporate artificial intelligence for EEG analysis, pattern recognition, music suggestions, and emotional tracking based on EEG data.

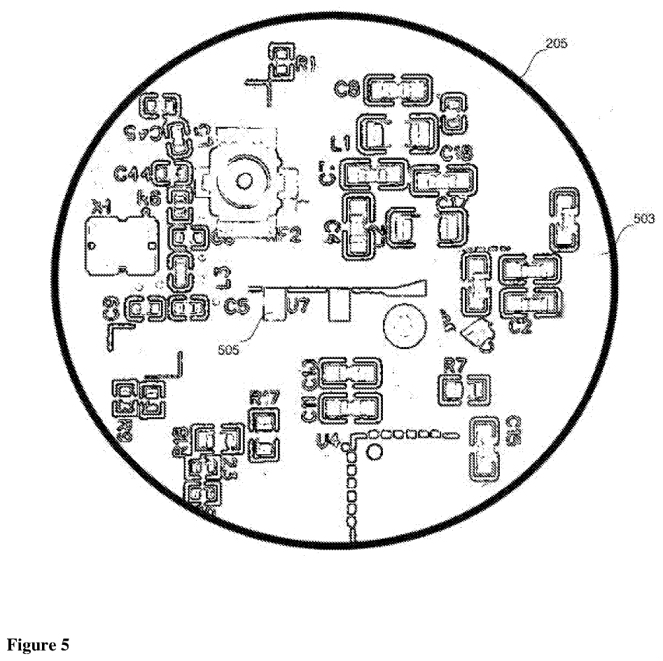

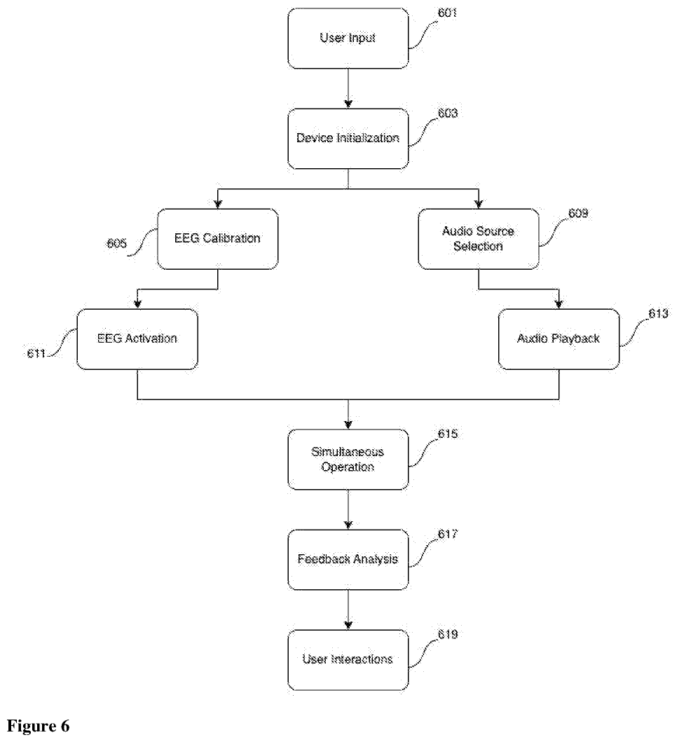

The technical outline included in the Circuitry in FIG. 5 depicts a suggested circuit, 205, defining the system's technical foundation. This circuit prioritizes the raw EEG data collection through carefully crafted electrode interfaces and emphasizes signal processing. The latter involves amplifying and digitizing the analog brainwave signals and conducting advanced frequency domain analysis. The design focuses on clear signal pathways and digital filtering techniques to uphold EEG data quality. Operational overview which has the Apparatus Flow Chart includes FIG. 6 provides a flowchart detailing the chronological user interaction with the earbuds.

The sequence initiates with 601—User Input, where the earbuds are activated and the desired audio content is selected. Earbud activation occurs once silicone ear tip 105 is placed directly into the ear canal. Following this, 603—Device Initialization checks the device's positioning and internal system operability. After passing this stage, the system gears up for EEG monitoring. Post-initialization, the 605—EEG Calibration phase adjusts the system based on the user's unique brainwave signatures, promoting consistent accuracy. The 609—Audio Source Selection allows for audio content choices, while 611—EEG Activation activates the electrodes for comprehensive brainwave monitoring, including but not limited to alpha, beta, and gamma waves. Real-time Feedback and Interaction is placed during 613—Audio Playback, the system showcases its dual capability of audio delivery and simultaneous brainwave monitoring. This live data is then relayed and visualized for the user. The 615 Feedback Analysis offers user feedback, suggesting audio adjustments or cues, enhancing the listening experience. Lastly, 619 Optional User Interactions offers users an avenue to modify their experience and access their EEG data, with potential auditory alerts based on the EEG analysis.

It should be noted that the system described herein may be modified in known or foreseeable manners, and the above description is not intended to be limiting. One or more aspects of this disclosure can meet certain objectives, while one or more aspects can lead to certain other objectives. The generic principles shown can be applied to various other embodiments without departing from embodiments of the disclosure. Such other modifications to the embodiments, examples, uses, are intended to be encompassed by the claims attached.

1. An earbud system, comprising:
a. a body containing audio playback components;
b. a silicone eartip that incorporates conductive filaments facilitating the capture of electroencephalography (EEG) signals;
c. a ground electrode positioned to make contact with an user's ear;
d. a battery unit to power the system;
e. a circuit integrated within the earbud system for EEG data acquisition, amplification, and digitization;
f. a communication module for wirelessly transmitting EEG and audio data.

2. The earbud system of claim 1, wherein the silicone eartip may be partitioned into a grid formation which enhances the surface area for the electrode arrays, thereby improving EEG signal reading accuracy.

3. The earbud system of claim 1, further comprising software compatibility to interface with Human-Machine Interface applications on external devices, enabling real-time visualization and analysis of EEG data.

4. The earbud system of claim 5, wherein the software utilizes artificial intelligence for EEG pattern recognition, emotional tracking, and personalized music recommendations based on the analyzed EEG data.

5. The earbud system of claim 1, further designed for applications in medical scenarios, including but not limited to open brain surgeries, to provide non-obtrusive real-time EEG monitoring.

6. The earbud system of claim 1, wherein said system aids in emotional balance, stress relief, and neurostimulation by providing feedback-based audio adjustments or cues.

7. The earbud system of claim 1, designed to function both for recreational audio playback and for health-monitoring purposes.

8. A method for capturing EEG data and audio playback using an earbud system, said method comprising the steps of:
a. ensuring correct positioning of the earbud for optimal EEG signal capture;
b. calibrating the system to the user's unique brainwave patterns;
c. selecting an audio source for playback;
d. activating EEG monitoring, wherein the system captures brainwave activities, including but not limited to alpha, beta, gamma, and theta waves;
e. providing real-time feedback to the user based on analyzed EEG data;
f. adjusting audio output based on user feedback and EEG data interpretation.

9. The method of claim 8, further comprising the step of wirelessly transmitting captured EEG data to an external device for further analysis and visualization.

10. The earbud system of claim 1, wherein the integration of EEG systems within the earbud results in a minimally invasive method for screening neurological conditions while the user benefits from standard earbud functionalities.

11. The earbud system of claim 1, wherein the system's purpose extends to wellness sectors, facilitating mood-based therapeutic interventions and entertainment sectors for content delivery based on real-time biometric feedback.

12. The earbud system of claim 1, wherein the system is designed ergonomically to prioritize user comfort while ensuring consistent and accurate EEG data capture.
